(19) United States
(12) Patent Application Publication
(10) Pub. No.: US 2026/0207901 A1
(43) Pub. Date: Jul. 23, 2026

(54) EAR CATHETER FOR APPLYING MEDICATION

(71) Applicant: Auriventis Medical GmbH, Lörrach (DE)

(72) Inventors: Holger SUDHOFF, Bielefeld (DE); Maximilian SUDHOFF, Bielefeld (DE); Aleksandar OBRADOVIC, Lörrach (DE); Milisav OBRADOVIC, Lörrach (DE)

(73) Assignee: Auriventis Medical GmbH, Lörrach (DE)

(21) Appl. No.: 19/144,526

(22) PCT Filed: Dec. 14, 2023
(86) PCT No.: PCT/EP2023/085939

§ 371 Date: Jun. 30, 2025

(30) Foreign Application Priority Data
Dec. 30, 2022 (DE) 10 2022 135 027.4

(51) Int. Cl. A61M 25/10

(52) U.S. Cl. CPC A61M 25/1011; A61M 2025/1015; A61M 2025/1061; A61M 2025/1093; A61M 2202/0007; A61M 2205/0266; A61M 2210/0668

(57) ABSTRACT

The invention relates to an ear catheter for insertion into the middle-ear region through an incision in the eardrum, comprising a catheter tube (2) having a distal end (d) and a proximal end (p), wherein the catheter tube (2) comprises at least three lumina, namely a first lumen in the form of an administration lumen (3), a second lumen in the form of an inflation lumen (4), and a third lumen in the form of a pressure-relief lumen (5), and wherein the ear catheter (1) further comprises a first distal sealing element (6) and a second sealing element (7), positioned proximally to the first sealing element (6), wherein the sealing elements (6, 7) in the distal region of the catheter tube (2) form an administration region and at least the first sealing element (6) is provided in the form of an inflatable balloon, which is connected to the inflation lumen (4) in a fluid-permeable manner. The ear catheter according to the invention allows disorders of the middle ear to be effectively treated.

The invention relates to an ear catheter for administering medication into the auditory tube, wherein the ear catheter can be placed into the middle-ear region in particular through an access point in the eardrum.

Numerous disorders of the middle and inner ear are known which require localised treatment. If a topical treatment needs to be given, this can take place both through the eardrum and also via the auditory tube. The inner ear can also be treated via the middle-ear region. For the treatment, an ear catheter inserted through the nose is often used, also to introduce medications. However, administering medications into the auditory tube or the middle ear, in particular in liquid form, is often not sustainable, since the medication generally runs into the nasopharyngeal space shortly after being administered.

The treatment of a range of infections and inflammations and, for example, the treatment of hearing loss or Meniere's disease require the longer-term administration of medications into the auditory tube or the middle ear, which can sometimes last for days or a few weeks. Typically, in these cases, new medication, for example a corticosteroid, is introduced at regular intervals. This treatment is time-consuming and requires regular and ongoing medical supervision and care.

For this reason, it would be desirable to have an ear catheter using which a medication can be administered into the middle ear and can be kept there for a longer period of time.

DE 10 2018 102 937 A1 describes an ear catheter which can be introduced into the auditory tube via the nasopharyngeal space and comprises an expandable balloon for sealing from the nasopharyngeal space. An injection channel extends through the balloon, such that, at the distal end of the balloon, a medication can be administered into the auditory tube and is prevented from flowing back through the balloon and into the nasopharyngeal space.

Administering the medication using the known ear catheter accordingly takes place non-invasively via the nasopharyngeal space and the auditory tube (the Eustachian tube).

However, it is not always easy, and sometimes impossible, to position an ear catheter through a patient's nose. Positioning it also requires some training. If the catheter consists of a very flexible material, an additional insertion aid often has to be used to position the catheter. This results in additional materials being used and in additional costs.

There are thus situations in which administering medication into the middle-ear region is advantageously carried out invasively through the eardrum and not through the nasopharyngeal space. Interventions through the eardrum in which a small incision is made in the eardrum, a paracentesis, have been being carried out for some time and, from experience, heal without complications. They are used, for example, when channelling accumulations of fluid out of the tympanic cavity.

One problem addressed by the invention is therefore to provide an ear catheter which has the option of administering medication and is suitable for an invasive application through the eardrum.

Another problem addressed by the invention is to provide an ear catheter which has the option of administering medication and can also be used without an additional insertion aid.

Not least, one problem addressed by the invention is to provide an ear catheter which has the option of administering medication and comprises a device which can keep the administered medication at the administration site for a longer period of time.

This problem is solved by an invention having the features of claims 1, 7 and 9. Advantageous configurations are found in the dependent claims. It should be noted that the features that are set out individually in the claims can also be combined with one another in any technologically viable manner, and thus demonstrate further configurations of the invention.

An ear catheter according to the invention comprises a catheter tube having a distal end and a proximal end, wherein, in a first embodiment, the catheter tube comprises at least three lumina, namely a first lumen in the form of an administration lumen, a second lumen in the form of an inflation lumen, and a third lumen in the form of a pressure-relief lumen. In a second embodiment, the ear catheter comprises only two lumina, namely a first lumen in the form of an administration lumen and a second lumen in the form of an inflation lumen. In a third embodiment, the ear catheter comprises only a first lumen in the form of an administration lumen.

In the context of the present invention, “distal” refers to the part of the device pointing away from the user along the ear catheter, while “proximal” refers to the part of the device pointing towards the user along the ear catheter. Further details on the positional relationships can be found in the drawings.

In the first embodiment, the catheter tube of the ear catheter preferably comprises at least three lumina, namely a first lumen in the form of an administration lumen, a second lumen in the form of an inflation lumen, and a third lumen in the form of a pressure-relief lumen.

The administration lumen extends through the catheter tube from the proximal end up to the administration region and opens to the outside in the administration region through an opening, the administration opening. The administration lumen is provided for administering a medication through the opening in the administration region.

The administration region is provided in the distal region or at the distal end of the catheter tube.

The administration lumen can optionally comprise a closure, for example a one-way valve, preferably in the form of a duckbill valve, which prevents the administered active substance from flowing out in the proximal direction through the administration lumen. The corresponding valve is preferably provided at the proximal end of the administration lumen, and in any case it is provided proximally to the administration opening.

It is alternatively also possible to seal off the administration lumen, which can consist of a soft plastics tube, by means of a second sealing element, provided that it is designed as a balloon, and thus bring about closure of the administration lumen.

To introduce a generally liquid active substance, the proximal end of the administration lumen is preferably provided with a conventional connection, in particular a Luer lock, to which a syringe for introducing the active substance solution can be coupled, for example.

In the first variant of the ear catheter, the administration region is delimited distally by a first sealing element and proximally to the first sealing element by a second sealing element.

The first sealing element is used to seal the middle-ear region from the nasopharyngeal space so that medications administered into the middle-ear region cannot flow out into this space, but instead remain at the site of action. The first sealing element is preferably provided in the form of a balloon, wherein only a minimal overpressure is applied to the balloon so that it closes the auditory tube. Accordingly, this first sealing element is only unfolded or inflated after positioning the catheter through the eardrum. The second sealing element is used to seal the middle-ear region from the outer ear such that, as far as possible, no administered medication can escape through the incision in the eardrum. The second sealing element can likewise be provided in the form of an inflatable balloon, but the second sealing element can also be provided as another seal, for example in the form of a sealing ring or another sleeve-like device, which is preferably made of silicone or another flexible, sealing material and is preferably provided to be movable, i.e. displaceable, along the catheter tube.

Preferably, the second sealing element is provided for sealing the eardrum on the outer side of the eardrum. In an alternative embodiment, the second sealing element can be provided in the form of a balloon for also sealing the eardrum on its inner side.

If the second sealing element is also provided in the form of an inflatable balloon, this second sealing element can also be connected to the inflation lumen in a fluid-permeable manner. This is advantageous because the first and second sealing elements are thus inflatable simultaneously by an inflation lumen, which makes handling simpler.

In the alternative embodiment comprising two inflatable balloons as the first and second sealing elements, the catheter tube can comprise two inflation lumina, wherein the first inflation lumen is connected to the first sealing element and the second inflation lumen is connected to the second sealing element.

The inflation lumen extends through the catheter tube from the proximal end up to the first sealing element and is connected to the first and optionally to the second sealing element in a fluid-permeable manner. The inflation lumen is provided for inflating and deflating the first and optionally the second sealing element.

At the proximal end of the catheter tube, the inlet of the inflation lumen and of the administration lumen can be different shapes, so that they do not get mixed up during use.

The pressure-relief lumen extends through the catheter tube from the proximal end up to the administration region and opens to the outside in the administration region. It is provided for relieving pressure in the application region or the middle ear.

So that no administered active substance can escape from the administration region to the outside as far as possible through the pressure-relief lumen, the pressure-relief lumen preferably comprises a valve or a valve-like device which both allows for pressure relief and prevents active substance from leaking out indiscriminately. This in particular means that no active substance is supposed to leak out through the pressure-relief lumen as far as possible, provided that an overpressure has not built up. A corresponding valve can accordingly be designed such that it merely covers the outlet opening in the pressure-relief lumen, but without requiring a significant overpressure to open.

The region of the catheter between the first and the second sealing element is referred to as the administration region. The administration region thus comprises the region of the catheter in which the medication is administered in the middle-ear region and can take effect in the inner-ear region or middle-ear region. Accordingly, the administration region is intended to comprise at least the region to be treated, and it is preferably intended to correspond to this as precisely as possible.

Once the first sealing element has reached the site within the ear, i.e. the part of the middle ear to be closed, for example the auditory tube, a liquid is applied to the balloon of the sealing element and this is expanded in a known manner.

To expand the balloon, a physiological saline solution preferably acts as the liquid. To introduce the liquid, the proximal end of the inflation lumen is provided with a conventional connection, in particular a Luer lock, to which a syringe for the saline solution can be coupled. The catheter tube is designed to have a corresponding valve, for example a one-way valve, such that the balloon maintains the pressure.

If two inflation lumina are provided, the proximal connections of both inflation lumina are provided with a conventional connection, in particular a Luer lock, to which a syringe for introducing the liquid can be coupled. The inflation lumina are designed to have corresponding valves, for example one-way valves and preferably duckbill valves, such that the balloons each maintain the pressure.

In this case, it is expedient to design the balloon(s) as low-pressure cuffs and to provide pressure limiters in the inflation lumen/lumina, which limit the pressure to a low pressure as a safety measure. Preferably, the valve is an umbrella valve. Standard materials can be used for the balloons and the catheter. These are plastics materials which are medically approved, for example PVC, polyamide, polyethylene, polypropylene and the like. The materials should be sufficiently resilient as to allow them to be inserted into the auditory tube.

According to a second embodiment, ear catheters are conceivable which comprise a tube in the form of a catheter tube having a distal end and a proximal end, comprising a first lumen in the form of an administration lumen and a second lumen in the form of an inflation lumen.

This embodiment substantially corresponds to the first variant, but a pressure-relief lumen is omitted. This is possible because, in this variant, a second, more proximally positioned sealing element is provided and any overpressure potentially building up in the middle ear can be decreased by the incision that has been made in the eardrum.

Accordingly, all the statements and comments made regarding the first embodiment are also applicable to this second embodiment provided that they relate in particular to the administration lumen, the inflation lumen and the first or distal sealing element.

According to a third embodiment, variants of the ear catheter are conceivable which comprise a tube in the form of a catheter tube having a distal end and a proximal end, comprising a first lumen in the form of an administration lumen and an additional administration device.

In this variant, the administration lumen is primarily not for directly administering a medication, but for receiving and positioning the administration device. The administration lumen extends through the catheter tube from the proximal end up to the administration region, where it opens to the outside by way of an opening. The opening in the administration lumen is preferably at the distal end of the administration lumen or the open distal end of the administration lumen forms this opening. It is thus possible to advance the administration device through the administration lumen, through the distal opening in the administration lumen, up to the administration site, for example in the middle ear.

Even if, in the case of this third embodiment, the administration lumen is not provided primarily for directly administering a medication and preferably does not have a corresponding closure, the administration lumen can still optionally be suitable for administering a medication, for example in order to introduce more medication to the administration element, which will be described in greater detail in the following.

The administration device can designed differently, but routinely comprises a generally tubular or wire-shaped introduction element, for example in the manner of an introduction wire or a guide wire, and an administration element provided at the distal end of the introduction element.

The administration element can be fixed to the introduction element via a coupling element, wherein the coupling element can also be part of the introduction element. Alternative embodiments are also conceivable in which the administration element itself is part of the introduction element or is directly, namely without a special coupling element, for example by bonding, welding or other known connection techniques, fixed to the introduction element.

The introduction element can be made of any material, wherein the material has to have sufficient rigidity that it is possible to advance the administration device in the administration lumen. Suitable materials are known to a person skilled in the art from the medical technology sector.

The distal end of the administration element or the introduction or coupling element should be designed to be atraumatic in order to prevent injury to the sensitive middle-ear region when it is being positioned. The distal end of the ear catheter per se should also be provided to be atraumatic in order to prevent injury when positioning the ear catheter.

The administration element is the actual carrier of the medication to be administered and serves to administer and deposit the medication in the middle-ear region. The administration element is thus suitable for receiving and then administering the medication to be administered.

Possible configurations and materials of the administration element can comprise woven textiles or non-woven fleece materials, structures made of shape-memory materials, or polymers such as wire meshes or coils as well as combinations of said configurations and materials. Administration elements are accordingly all of those structures which are suitable for receiving a medication and then providing it for administration and administering it to the middle ear at least over a certain time period.

Structures according to the invention made of shape-memory materials or polymers can also be wires, stranded wires, thread meshes or spirals, for example, which are suitable for receiving a substantially liquid or pasty medication. It is also conceivable for corresponding meshes or spirals to be directly coated with a medication or a medication-carrying coating, for example in the form of a specific coating which carries the medication or in which the medication is embedded.

Further suitable combinations for an administration element of this kind are, for example, with a textile coating or fleece or wires, meshes or spirals provided with a sponge-like or porous plastics material.

Textiles or fleece or sponge-like or porous plastics material, which are potentially additionally arranged in a kind of cage, are also conceivable as the administration element, wherein this cage is for example made of shape-memory materials or materials having shape-memory properties.

Medication-coated balloons or medication-administering balloon reservoirs are conceivable administration elements.

Depending on the type of administration element, the administration lumen can also be used to introduce new medication to the administration element. This is useful in administration elements of this kind which can receive new medication in the manner of a reservoir, therefore, for example, administration elements having sponge-like properties, balloons or meshes.

In an alternative variant of this third embodiment, a first sealing element can be provided at the distal end of the ear catheter, wherein, according to this embodiment, the first sealing element is preferably formed by one or more fins, which are preferably arranged annularly at the distal end of the administration element and are designed to be flexible such that they can seal the administration region from the oral-pharyngeal space in a fluid-tight manner.

A fin-like sealing element of this kind can assume a compressed state in the administration lumen and, when released, can assume an expanded state around the middle ear, which has a greater diameter than the compressed state.

In this kind of configuration of the distal sealing element comprising a fin structure, complications can arise when removing the ear catheter through the eardrum, since, in particular, a distal sealing element provided in the manner of fins cannot easily be transferred back into the compressed state from the expanded state and thus retracted into the administration lumen.

Accordingly, in embodiments which in particular have a fin-like distal sealing element, a preferably thread-like or wire-like retrieval element, at which the ear catheter can alternatively also be removed through the oral-pharyngeal space, is optionally provided at the distal end of the ear catheter.

For the balloons and catheters in all the embodiments, standard materials can be used as are known in medical technology for catheters or for balloon catheter technology. These are in particular plastics materials which are medically approved, for example PVC, polyamide, polyethylene, polypropylene and the like. The materials should be sufficiently resilient as to allow them to be inserted into the middle ear through the eardrum.

The ear catheter according to the invention is particularly suitable for insertion of antibiotic solutions through the eardrum into the middle-ear region, which is useful for persistent bacterial infections. It can also be useful to carry out the standard treatment of hearing loss, which is done with a corticosteroid, via an ear catheter of this kind.

In all variants and embodiments, by continuously providing the active substance, the ear catheter according to the invention often allows for a much more intensive and shorter treatment duration, even in just one treatment, and also in patients in whom access is not possible through the nasopharyngeal space, but instead has to be through the eardrum. The treatment duration is thus also shortened for these patients, and in some circumstances to a few days.

Furthermore, anti-inflammatory medications can be inserted via the ear catheter.

After the treatment, the ear catheter can be removed from the ear again, where necessary once pressure has been relieved and the balloons have been deflated.

The ear catheter also allows for treatment of disorders of the inner ear, since the medications can generally pass through the membrane of the round window into the inner ear. This is a diffusion process.

The invention also relates to a method for administering a medication through the eardrum into the middle ear by means of an ear catheter according to the invention, comprising the following steps:
(A) making an incision in the eardrum; and
(B) inserting an ear catheter according to the invention through the incision in the eardrum; and
(C) positioning the ear catheter in the middle ear; and
(D) optionally inflating the first and/or second sealing element; and
(E) optionally administering the medication or inserting the administration device through the administration lumen; and
(F) optionally administering further medication through the administration lumen; and
(G) optionally deflating the first and/or second sealing element; and
(H) optionally carrying out additional pressure relief; and
(I) removing the ear catheter.

The invention and the technical field are described in greater detail in the following with reference to the drawings. It should be noted that the drawings show a particularly preferred variant of the invention; however, the invention is not limited to the variant shown. Provided that it is technically viable, the invention in particular includes any combination of the technical features that are set out in the claims or are described in the description as being relevant to the invention.

### IN THE DRAWINGS

FIG. 1 shows the access point for an ear catheter according to the invention through an incision in the eardrum;

FIG. 2 shows a first embodiment of the ear catheter according to the invention;

FIG. 3 shows the first embodiment of the ear catheter according to the invention with an alternative selection of the second sealing element;

FIG. 4 shows the second embodiment of the ear catheter according to the invention with just one first sealing element;

FIG. 5 shows the third embodiment of the ear catheter according to the invention without sealing elements but with an administration element; and

FIG. 6 shows a variant of the third embodiment of the ear catheter according to the invention with a first sealing element.

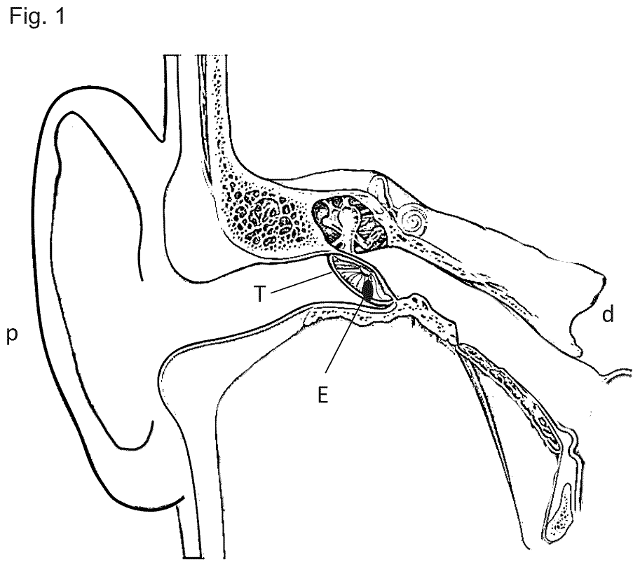

FIG. 1 schematically shows the human ear having the eardrum T and the site of the incision E for introducing the ear catheter according to the invention.

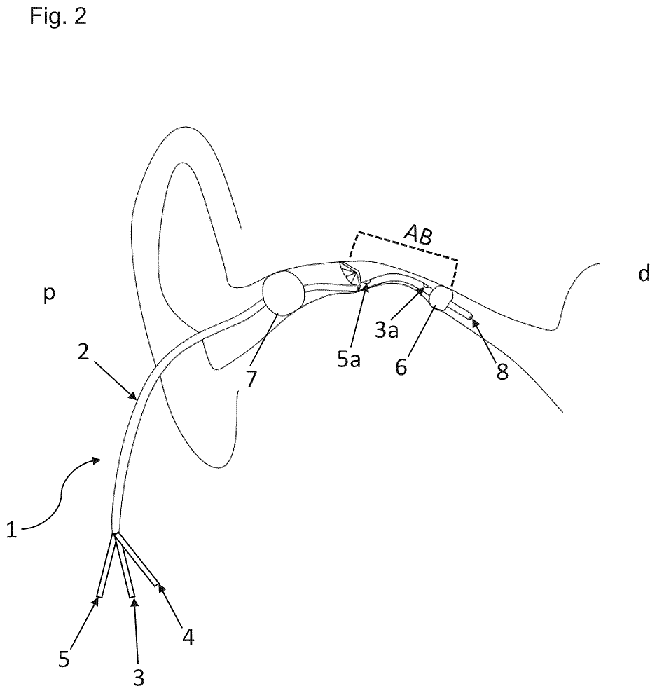

FIG. 2 shows a first embodiment of the ear catheter 1 according to the invention comprising a catheter tube 2 having an administration lumen 3, an inflation lumen 4 and a pressure-relief lumen 5. The catheter tube 2 comprises an atraumatically shaped tip 8 at the distal end in order to prevent injury to the sensitive middle ear when positioning the ear catheter 1. A first sealing element 6 in the form of an inflatable balloon, which is connected to the inflation lumen 4 in a fluid-permeable manner, is provided at the distal end of the catheter tube 2. A second sealing element 7 in the form of an inflatable balloon, which is likewise connected to the inflation lumen 4 in a fluid-permeable manner, is positioned more proximally, and in the positioned state, in the outer ear canal outside the middle ear. In an alternative embodiment, a separate inflation lumen 4 can be provided for each of the sealing elements 6, 7.

The first and the second sealing element 6, 7 form an administration region AB and an active substance can be administered into the administration region AB via the administration lumen 3 through an opening 3a in the administration lumen 3. The overpressure resulting from administering the active substance in the administration region AB is equalised by an opening 5a in the pressure-relief lumen 5. So that no active substance can escape through the administration lumen 3 or the pressure-relief lumen 5, these lumina can comprise corresponding valves.

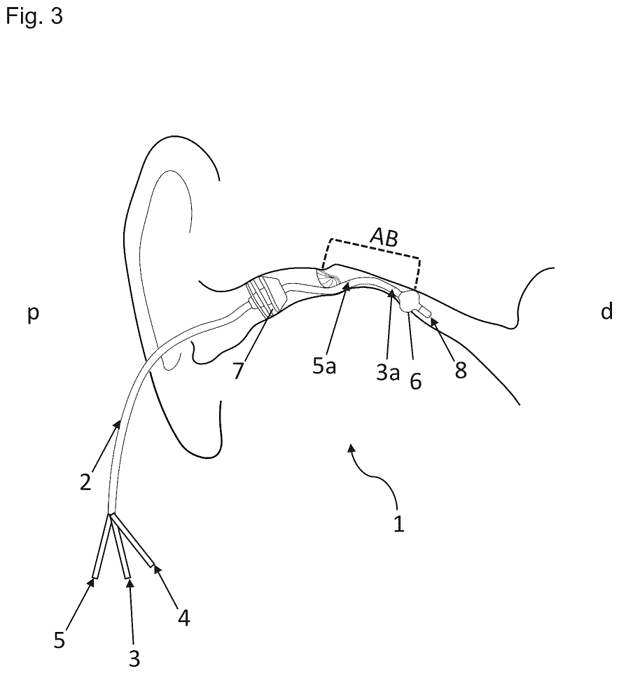

FIG. 3 shows a second embodiment of the ear catheter 1 according to the invention comprising a catheter tube 2 having an administration lumen 3, an inflation lumen 4 and a pressure-relief lumen 5. The catheter tube 2 comprises an atraumatically shaped tip 8 at the distal end in order to prevent injury to the sensitive middle ear when positioning the ear catheter 1. A first sealing element 6 in the form of an inflatable balloon, which is connected to the inflation lumen 4 in a fluid-permeable manner, is provided at the distal end of the catheter tube 2.

A second sealing element 7 in the form of a closure arranged annularly around the catheter tube 2 is positioned more proximally, and in the positioned state, in the outer ear canal outside the middle ear. This second sealing element 7 is preferably made of silicone or another sealing and simultaneously flexible material. The diameter of this second sealing element 7 is selected such that there is at least one form fit between the second sealing element 7 and the outer ear canal. In this embodiment, this second sealing element 7 can also comprise a plurality of serial annular or disc-shaped elements, as shown.

In this embodiment too, the first and the second sealing element 6, 7 form an administration region AB and an active substance can be administered into the administration region AB via the administration lumen 3 through an opening 3a in the administration lumen 3. The overpressure resulting from administering the active substance in the administration region AB is equalised by an opening 5a in the pressure-relief lumen 5. So that no active substance can escape through the administration lumen 3 or the pressure-relief lumen 5, these lumina can comprise corresponding valves.

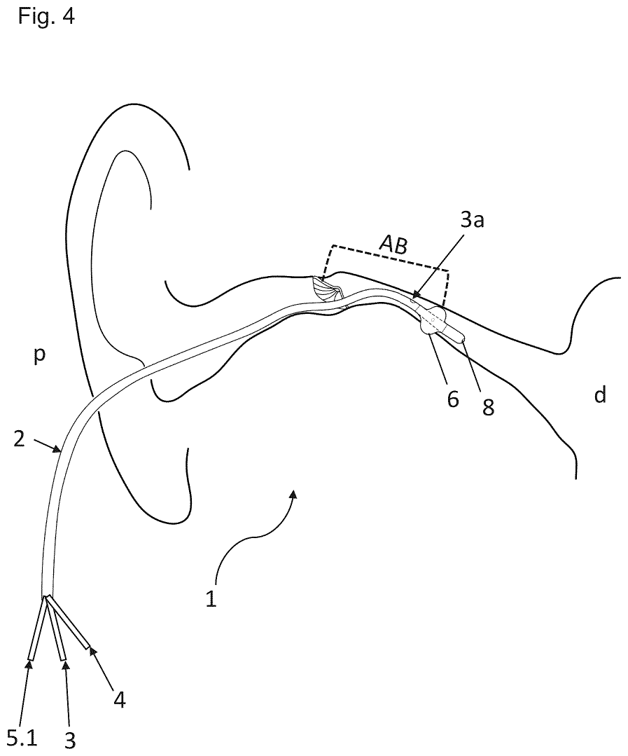

FIG. 4 shows a third embodiment of the ear catheter 1 according to the invention comprising a catheter tube 2 having an administration lumen 3 and an inflation lumen 4. The catheter tube 2 comprises an atraumatically shaped tip 8 at the distal end in order to prevent injury to the sensitive middle ear when positioning the ear catheter 1. A first sealing element 6 in the form of an inflatable balloon, which is connected to the inflation lumen 4 in a fluid-permeable manner, is provided at the distal end of the catheter tube 2. This embodiment does not comprise a second sealing element more proximally.

In this embodiment, the administration region AB is formed by the first sealing element 6 and the eardrum, wherein a second sealing element is omitted and an excessive amount of medication is prevented from leaking out of the eardrum by the most precise possible form fit between the catheter tube 2 and the eardrum incision. Into the administration region AB an active substance can be administered into the administration region AB via the administration lumen 3 through an opening 3a in the administration lumen 3. The overpressure resulting from administering the active substance in the administration region AB can escape through the incision in the eardrum. Therefore, in the present case, a pressure-relief lumen can be omitted, and a corresponding lumen 5.1 can optionally be used as a guidewire lumen. So that no active substance can escape through the administration lumen 3, the administration lumen 3 can comprise a corresponding one-way valve, which is preferably arranged proximally.

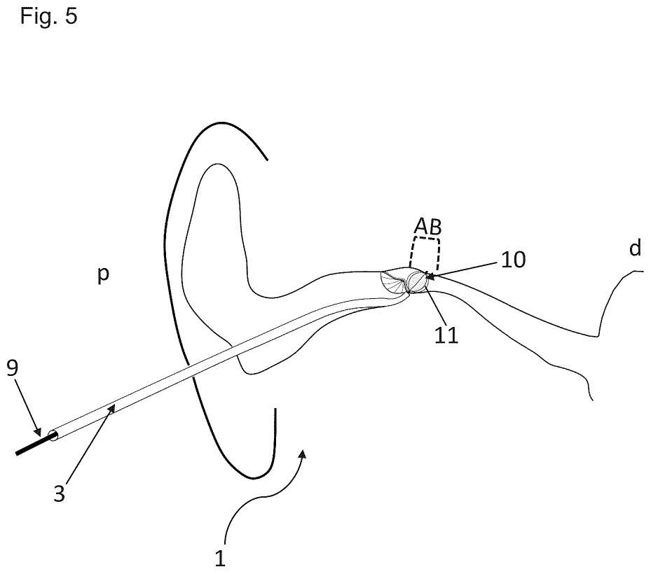

FIG. 5 shows another embodiment of the ear catheter 1 according to the invention comprising a catheter tube 2 having an administration lumen 3. This embodiment does not comprise any sealing elements, since the medication is administered by an administration element 10 which is suitable for retaining the medication over a longer time period and administering it to the middle ear. The administration element 10 is provided at the distal end of an introduction element 9, by means of which the administration element 10 can be advanced through the administration lumen 3 to the administration region AB. As shown in the present case, the administration element 10 can be connected to the introduction element 9 via a coupling element 11. In the present case, the coupling element 11 is provided in the form of a nitinol basket, which surrounds the administration element 10 and accordingly assumes a folded-up state within the administration lumen 3 and reversibly assumes an expanded state outside the administration lumen 3.

In the embodiment shown, the administration element 10 is provided in the form of a sponge-like element which is suitable for receiving a medication. The medication can optionally also be introduced to the administration element 10 via the administration lumen 3 after positioning the administration element 10. This can be useful because the administration element 10 also assumes a compressed state within the administration lumen 3 and accordingly can optionally only receive a smaller quantity of medication compared with the expanded state outside the administration lumen 3.

In this embodiment, the administration region AB substantially corresponds to the length of the administration element 10 which administers the medication to the middle ear.

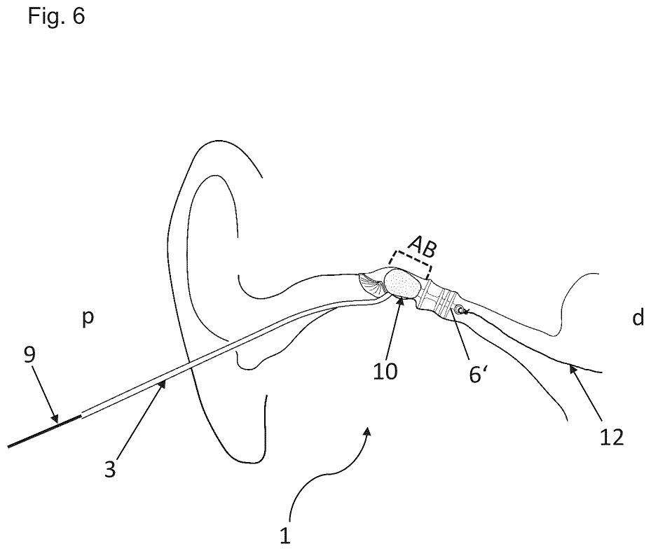

FIG. 6 shows another embodiment of the ear catheter 1 according to the invention comprising a catheter tube 2 having an administration lumen 3. This embodiment comprises a distal sealing element 6′, which is formed by one or more fins, which are preferably arranged annularly and at the distal end of the administration element 10 and are designed to be flexible such that they can seal the administration region AB from the oral-pharyngeal space in a fluid-tight manner.

The administration element 10 is provided at the distal end of an introduction element 9, by means of which the administration element 10 can be advanced through the administration lumen 3 to the administration region AB. As shown in the present case, the administration element 10 can be directly connected to the introduction element 9 by bonding or other techniques, for example.

In the embodiment shown, the administration element 10 is provided in the form of a sponge-like element which is suitable for receiving a medication. The medication can optionally also be introduced to the administration element 10 via the administration lumen 3 after positioning the administration element 10. This can be useful because the administration element 10 also assumes a compressed state within the administration lumen 3 and accordingly can optionally only receive a smaller quantity of medication compared with the expanded state outside the administration lumen 3.

In this embodiment, the administration region AB substantially corresponds to the length of the administration element 10 which administers the medication to the middle ear.

Since, in this embodiment, the distal sealing element 6′is formed by one or more fins, it can be useful not to remove the ear catheter 1 through the incision in the eardrum, but instead through the nasopharyngeal space. For this purpose, a retrieval element 12, for example in the form of a thread, by which the catheter 1 can be grasped and removed through the oral and nasal cavity, is provided at the distal end of the catheter 1.

Optionally, it is also useful to position this embodiment through the oral-pharyngeal space and to only guide the catheter tube 2 having the administration lumen 3 through the eardrum to administer fresh medication.

### LIST OF REFERENCE SIGNS

1 Ear catheter
2 Catheter tube
3 Administration lumen (3a opening in the administration lumen, administration opening)
4 Inflation lumen
5 Pressure-relief lumen (5a opening in the pressure-relief lumen)
6 First sealing element
7 Second sealing element
8 Atraumatic tip
9 Introduction element
10 Administration element
11 Coupling element
12 Retrieval element
AB Administration region
p Proximal
d Distal

1. Ear catheter for insertion into the middle-ear region through an incision in the eardrum, comprising a catheter tube (2) having a distal end (d) and a proximal end (p), wherein the catheter tube (2) comprises at least three lumina, namely a first lumen in the form of an administration lumen (3), a second lumen in the form of an inflation lumen (4), and a third lumen in the form of a pressure-relief lumen (5), and wherein the ear catheter (1) further comprises a first distal sealing element (6) and a second sealing element (7), positioned proximally to the first sealing element (6), wherein the sealing elements (6, 7) in the distal region of the catheter tube (2) form an administration region and at least the first sealing element (6) is provided in the form of an inflatable balloon, which is connected to the inflation lumen (4) in a fluid-permeable manner.

2. Ear catheter according to claim 1, wherein the second sealing element (7) is provided in the form of an inflatable balloon.

3. Ear catheter according to claim 1, wherein the second sealing element (7) is connected to the inflation lumen (4) in a fluid-permeable manner.

4. Ear catheter according to claim 1, wherein the catheter tube comprises a second inflation lumen and the second sealing element (7) is connected to the second inflation lumen in a fluid-permeable manner.

5. Ear catheter according to claim 1, wherein the second sealing element (7) is provided in the form of a sealing ring around the catheter tube.

6. Ear catheter according to claim 5, wherein the second element (7) is provided to be displaceable along at least part of the catheter tube (2).

7. Ear catheter for insertion into the middle-ear region through an incision in the eardrum, comprising a catheter tube (2) having a distal end (d) and a proximal end (p), wherein the catheter tube (2) comprises only a two lumina, namely a first lumen in the form of an administration lumen (3) and a second lumen in the form of an inflation lumen (4), and wherein the ear catheter (1) further comprises only a first distal sealing element (6) and the first sealing element (6) is provided in the form of an inflatable balloon, which is connected to the inflation lumen (4) in a fluid-permeable manner.

8. Ear catheter according to claim 1, wherein the catheter tube (2) comprises an atraumatically shaped tip (8) at the distal end.

9. Ear catheter for insertion through the eardrum into the middle-ear region, comprising a catheter tube (2) having a distal end and a proximal end and an administration device comprising and introduction element (9) and an administration element (10) provided distally on the introduction element (9), wherein the catheter tube (2) comprises a lumen reaching from the proximal end to the distal end in the form of an administration lumen (3) and wherein the administration lumen (3) is provided for inserting the administration device from the proximal end of the catheter tube (2), through the administration lumen (3), up to the distal end of the catheter tube (2).

10. Ear catheter accordingly to claim 9, wherein the administration element (10) is suitable for applying and depositing a medication in the middle-ear region and comprises woven textiles or non-woven fleece materials, structures made of shape-memory materials, or polymers such as wire meshes or coils as well as combinations of said configurations and materials.
